(19) United States
(12) Patent Application Publication
(10) Pub. No.: US 2025/0173794 A1
(43) Pub. Date: May 29, 2025

(54) AI Processing of Images to Generate an Insurance Recommendation

(71) Applicant: Cube Click, Inc., New York, NY (US)

(72) Inventors: Andrea Cambria, New York, NY (US); Richard Ricci, New York, NY (US)

(73) Assignee: Cube Click, Inc., New York, NY (US)

(21) Appl. No.: 18/385,128

(22) Filed: Oct. 30, 2023

Related U.S. Application Data
(63) Continuation-in-part of application No. 17/162,985, filed on Jan. 29, 2021, now Pat. No. 12,008,749.

(51) Int. Cl. G06Q 40/08; G06T 7/00; G16H 10/60; G16H 30/40

(52) U.S. Cl. CPC G06Q 40/08; G06T 7/0012; G16H 10/60; G16H 30/40; G06T 2207/30004

(57) ABSTRACT

The field of the invention relates to a system or method of using Artificial Intelligence (AI) processing of images to generate an insurance recommendation. The invention is configured to receive a preprocessed AI image associated with an insurance data and then compare and process the preprocessed AI image with a separate second or multiple AI algorithm. The invention is further configured to use a user adjustable probability ratio to identify at least one of: an anatomy identification, a pathology identification. The second or multiple processing of the preprocessed image may occur at an offsite location, such an insurance platform or the cloud. The second or multiple AI algorithm may generate an insurance recommendation based on the preprocessed AI image.

### CLAIM OF PRIORITY

This application claims priority to U.S. provisional application 62/955,321, filed on Dec. 30, 2019 for U.S. Patent No. U.S. Pat. No. 10,937,160. The contents of said provisional application and Patent No. U.S. Pat. No. 10,937,160 are hereby incorporated by reference in their entirety.

### FIELD OF THE INVENTION

The field of invention disclosed herein relates to a system or method utilizing Artificial Intelligence (AI) for generating an insurance recommendation. The system processes AI processed images obtained from users or providers using advanced AI algorithms. Wherein an AI processed image may be a medical or dental image. The algorithms may be executed in any order and include a range of instructions associated with a least one patient data. Some exemplary AI algorithm instructions for AI generation of an insurance recommendation may include:

(Example 1 Step A) Receiving, using at least one processing device, at least one AI processed image associated with at least one insurance data, wherein the one AI processed image is based on a first AI model which includes at least one of: an anatomy identification, a pathology identification.
(Example 1 Step B) Retrieving, using a storage device, at least one of: an anatomy dataset, a pathology dataset, and an insurance dataset.
(Example 1 Step C) Comparing, using the at least one processing device, the at least one AI processed image associated with the at least one insurance data with a second AI model based on at least one of: the anatomy dataset, the pathology dataset, and the insurance dataset, wherein the second AI model may generate a weighted probability ratio of at least one of: an anatomy identification, a pathology identification found in the at least one AI processed image. Wherein the second AI model is further configured for determining at least one insurance recommendation based on the weighted probability ratio of at least one of: the anatomy identification, the pathology identification found in the at least one AI processed image associated with the at least one insurance data.
(Example 1 Step D) Storing, using the storage device at least one of: the at least one insurance data, the at least one AI processed image, the weighted probability ratio of at least one of: the anatomy identification, the pathology identification found in the at least one AI processed image, and the at least one insurance recommendation.
(Example 1 Step E) Transmitting, using the at least one processing device at least one of: the at least one insurance data, the at least one AI processed image, the weighted probability ratio of at least one of: the anatomy identification, the pathology identification found in the at least one AI processed image, and the at least one insurance recommendation.

Another exemplary AI algorithm instructions for AI generation of an insurance recommendation may include:

(Example 2 Step A) Receive, using a processing device, at least one insurance data associated with at least one AI processed image. Wherein the processing device may execute an instruction in any order.

Wherein the one AI processed image is based on a first AI model which includes at least one of: an anatomy identification, a pathology identification.

(Example 2 Step B) Retrieve, using a storage device, at least one of: an anatomy dataset a, pathology dataset, an insurance dataset.
(Example 2 Step C) Compare, using the processing device, the at least one insurance data and the at least one AI processed image with a second AI model based on at least one of: the anatomy dataset, the pathology dataset, and the insurance dataset. Wherein the second AI model may generate a weighted probability ratio of at least one of: the anatomy identification, the pathology identification found in the at least one AI processed image.
(Example 2 Step D) Using the processing device, the second AI model is configured for determining the at least one insurance recommendation based on the weighted probability ratio of at least one of: an anatomy identification, a pathology identification found in the at least one AI processed image and associate with the at least one insurance data associated with the at least one AI processed image.
(Example 2 Step E) Store, using the storage device at least one of: the at least one insurance data, the at least one AI processed image, the weighted probability ratio of at least one of: the anatomy identification, the pathology identification found in the at least one AI processed image, and the at least one insurance recommendation.
(Example 2 Step F) Transmit, using the processing device at least one of: the at least one insurance data, the at least one AI processed image, the weighted probability ratio of at least one of: the anatomy identification, the pathology identification found in the at least one AI processed image, and the at least one insurance recommendation.
And yet another exemplary AI algorithm instructions for AI generation of an insurance recommendation may include:
(Example 3 Step A) Receive, using at least one processing device, at least one insurance data associated with at least one AI processed image. Wherein the one AI processed image is based on a first AI model which includes at least one of: an anatomy identification, a pathology identification.
(Example 3 Step B) Retrieve, using a storage device, at least one of: an anatomy dataset a pathology dataset, an insurance dataset.
(Example 3 Step C) Compare, using the at least one processing device, the at least one insurance data and the at least one AI processed image with a second AI model based on at least one of: an anatomy dataset a pathology dataset, and an insurance dataset.
(Example 3 Step D) Generate using the second AI model a weighted probability ratio, with a user adjustable weighted probability ratio to produce of at least one of: a weighted probability ratio of an anatomy identification, a weighted probability ratio of a pathology identification found in the at least one AI processed image.
(Example 3 Step E) Generate using the second AI model at least one insurance recommendation based on the user adjustable weighted probability ratio of at least one of: a weighted probability ratio of an anatomy identification, a weighted probability ratio of a pathology identification found in the at least one insurance data associated with the at least one AI processed image.
(Example 3 Step F) Store, using the storage device at least one of: the at least one insurance data, the at least one AI processed image, the weighted probability ratio of at least one of: the weighted probability ratio of an anatomy identification, the weighted probability ratio of a pathology identification found in the at least one AI processed image, and the at least one insurance recommendation.
(Example 3 Step G) Transmit, using the at least one processing device at least one of: the at least one insurance data, the at least one AI processed image, the weighted probability ratio of at least one of: the weighted probability ratio of an anatomy identification, the weighted probability ratio of a pathology identification found in the at least one AI processed image, and the at least one insurance recommendation.

### BACKGROUND OF THE INVENTION

Digital imaging has revolutionized the field of medicine and dentistry. It has brought about significant advancements in patient care and treatment. With the widespread adoption of digital images, medical and dental clinics have embraced the benefits of this technology. Today, health care providers and staff are extensively trained in capturing and processing digital patient images, making them an integral part of routine procedures. Compared to the prior generation of imaging, that included x-ray film imaging, digital imaging offers numerous advantages. Firstly, digital images can be processed at a much faster rate, allowing the health care professionals to swiftly review and analyze patient data. This enhanced efficiency translates into improved productivity and reduced waiting times for patients. Furthermore, digital imaging significantly reduces a patient's radiation exposure, promoting a safer and more comfortable experience during medical or dental procedures.

The advent of digital imaging has paved the way for comprehensive patient image management services. These services encompass a wide range of applications, such as offsite image hosting, seamless integration of images with insurance claims. Currently, many AI algorithms are used to assist healthcare providers with patient diagnosing. However, these AI processed images are currently not associated with a patient's insurance claim. There is a need for a platform to be developed to transmit, these AI processed images associated with a patient's insurance claim. Further, there is a need for the AI processed images associated with a patient's insurance to be further processed with a second or multiple AI algorithm in order for a third party to evaluate them. The second or multiple AI image algorithms would be able to compare a diagnosis form first AI algorithm to a second or multiple AL algorithm(s). Hence creating an AI algorithm check and balance system. A third party may include an insurance organization or claims processing service.

The healthcare industry is witnessing a paradigm shift towards integrated solutions that leverage digital imaging for enhanced patient care. This trend is driven by a growing awareness of the advantages offered by advanced imaging technologies in diagnosis and treatment planning. The demand for seamless integration of AI-processed images with insurance claims processing is inevitable. Hence reflecting a broader shift towards data-driven healthcare practices. Further, existing practices in managing patient images for insurance claims often face hurdles in terms of compatibility, data security, and streamlined workflows. These challenges impede the efficient utilization of digital imaging technologies in insurance processes, creating a clear need for a unified platform that addresses these issues.

The proposed integration of AI-processed images with insurance claims promises a host of benefits. It would significantly enhance the accuracy and efficiency of claims processing. Reducing the likelihood of errors and facilitating faster decision-making. Additionally, the integrated platform would lead to cost savings and improved resource allocation for healthcare providers and insurers alike. Consider, for instance, a scenario where a dental clinic employs AI-processed images for patient diagnosis. By seamlessly integrating these images with insurance claims, the clinic can expedite the claims process while ensuring accurate assessment. This not only leads to improved patient satisfaction but also optimizes resource allocation for both the clinic and the insurer.

Given the undeniable societal and professional demand for this technology, there is a pressing need for the development and implementation of an image platform that can accept an AI processed image are associated with a patient's insurance claim and then compare it to a second AI platform to generate an insurance recommendation.

### SUMMARY OF THE INVENTION

The present invention and its embodiments relates to AI Processing of Images to Generate an Insurance Recommendation. Wherein AI processing may include Machine Learning (ML) processing. Wherein AI processing may include deep learning processing. The system or method may include may use AI algorithms to calculate an insurance recommendation. The system or method may include at least one of: at least one of: a server, a processor, a microprocessor, a processing device. At least one of: a server, a processor, a microprocessor, a processing device may be configured to receive an AI image of a patient from a user, a provider, an e-commerce organization, a ML entity, an algorithm. Wherein an image may obtained from an image capture device. An example of a user or provider may include a patient, a dentist, a physician, a health care provider, an insurance company, a bioinformatics organization, a business, an e-commerce organization, a ML entity, an algorithm, a cloud based storage service, among others. At least one of: a server, a processor, a microprocessor, a processing device may be configured to receiving, using at least one processing device, at least one AI processed image associated with at least one insurance data, wherein the one AI processed image is based on a first AI model which includes at least one of: an anatomy identification, a pathology identification. Retrieving, using a storage device, at least one of: an anatomy dataset, a pathology dataset, and an insurance dataset. Comparing, using the at least one processing device, the at least one AI processed image associated with the at least one insurance data with a second AI model based on at least one of: the anatomy dataset, the pathology dataset, and the insurance dataset, wherein the second AI model may generate a weighted probability ratio of at least one of: an anatomy identification, a pathology identification found in the at least one AI processed image. Wherein the second AI model is further configured for determining at least one insurance recommendation based on the weighted probability ratio of at least one of: the anatomy identification, the pathology identification found in the at least one AI processed image associated with the at least one insurance data. Storing, using the storage device at least one of: the at least one insurance data, the at least one AI processed image, the weighted probability ratio of at least one of: the anatomy identification, the pathology identification found in the at least one AI processed image, and the at least one insurance recommendation. Transmitting, using the at least one processing device at least one of: the at least one insurance data, the at least one AI processed image, the weighted probability ratio of at least one of: the anatomy identification, the pathology identification found in the at least one AI processed image, and the at least one insurance recommendation. At least one of: the at least one insurance data, the at least one AI processed image, the weighted probability ratio of at least one of: the anatomy identification, the pathology identification found in the at least one AI processed image, and the at least one insurance recommendation may be a displayed on a user interface such as a GUI.

In another embodiment of the present invention the system or method may include at least one of: a server, a processor, a microprocessor, a processing device for providing an insurance recommendation of a patient's image with AI algorithms is described. At least one of: a server, a processor, a microprocessor, a processing device may include a computer vision component configured to analyze a patient's image, a memory configured to store instructions associated with at least one of: a server, a processor, a microprocessor, a processing device that may be coupled with a computer vision component and the memory. At least one of: a server, a processor, a microprocessor, a processing device may execute the instructions associated with a AI algorithm. At least one of: a server, a processor, a microprocessor, a processing device may process an instruction in any order. At least one of: a server, a processor, a microprocessor, a processing device may include image processing algorithms or an image processing engine. The image processing algorithms or the image processing engine may be configured to receive an image of a patient from at least one of: a user, a provider. An example of an image user or provider may include a patient, a dentist, a, an insurance company, a bioinformatics organization, a business, an e-commerce organization, a ML entity, an algorithm, a cloud based storage service, among others.

In another embodiment, the system or method may provide AI Processing of Images to Generate an Insurance Recommendation. The system or method may comprise at least one of: a server, a processor, a processing device that may be configured to receive, using a processing device, at least one insurance data associated with at least one AI processed image. Wherein the processing device may execute an instruction in any order. Wherein the one AI processed image is based on a first AI model which includes at least one of: an anatomy identification, a pathology identification. Retrieve, using a storage device, at least one of: an anatomy dataset a pathology dataset, an insurance dataset. Compare, using the processing device, the at least one insurance data and the at least one AI processed image with a second AI model based on at least one of: the anatomy dataset, the pathology dataset, and the insurance dataset. Wherein the second AI model may generate a weighted probability ratio of at least one of: the anatomy identification, the pathology identification found in the at least one AI processed image. Using the processing device, the second AI model is configured for determining the at least one insurance recommendation based on the weighted probability ratio of at least one of: an anatomy identification, a pathology identification found in the at least one insurance data associated with the at least one AI processed image. Store, using the storage device at least one of: the at least one insurance data, the at least one AI processed image, the weighted probability ratio of at least one of: the anatomy identification, the pathology identification found in the at least one AI processed image, and the at least one insurance recommendation. Transmit, using the processing device at least one of: the at least one insurance data, the at least one AI processed image, the weighted probability ratio of at least one of: the anatomy identification, the pathology identification found in the at least one AI processed image, and the at least one insurance recommendation.

Furthermore, a cluster analysis of the patient dataset may be performed with a cluster dataset or an algorithm to produce a correlation dataset or a correlated patient information. Moreover the correlation dataset or the correlated patient information may be provided to a user, a provider, an e-commerce organization, a ML entity, an algorithm. A user or a provider may also use the correlation dataset or the correlated information as a diagnostic aid which may be provided to a graphic user interphase (GUI) and/or a user interphase (UI).

In yet another embodiment of the present invention the system or method for providing AI processing of images to generate an insurance recommendation is described. The system or method may include receiving a patient's image from an image provider. An example of an image provider may include: a patient, a dentist, a physician, a health care provider, an insurance organization, a bioinformatics organization, an e-commerce service, a ML entity, an algorithm, a cloud based storage service, among others. A patient's image may next be processed with at least one of: a server, a processor, a microprocessor, a processing device. The processor may be configured to: execute an instruction in any order and receiving using at least one processing device, at least one insurance data associated with at least one AI processed image. Wherein the one AI processed image is based on a first AI model which includes at least one of: an anatomy identification, a pathology identification. Retrieve, using a storage device, at least one of: an anatomy dataset a pathology dataset, an insurance dataset. Compare, using the at least one processing device, the at least one insurance data and the at least one AI processed image with a second AI model based on at least one of: an anatomy dataset a pathology dataset, and an insurance dataset. Generate using the second AI model a weighted probability ratio, with a user adjustable weighted probability ratio to produce of at least one of: a weighted probability ratio of an anatomy identification, a weighted probability ratio of a pathology identification found in the at least one AI processed image. Generate using the second AI model at least one insurance recommendation based on the user adjustable weighted probability ratio of at least one of: a weighted probability ratio of an anatomy identification, a weighted probability ratio of a pathology identification found in the at least one insurance data associated with the at least one AI processed image. Store, using the storage device at least one of: the at least one insurance data, the at least one AI processed image, the weighted probability ratio of at least one of: the weighted probability ratio of an anatomy identification, the weighted probability ratio of a pathology identification found in the at least one AI processed image, and the at least one insurance recommendation. Transmit, using the at least one processing device at least one of: the at least one insurance data, the at least one AI processed image, the weighted probability ratio of at least one of: the weighted probability ratio of an anatomy identification, the weighted probability ratio of a pathology identification found in the at least one AI processed image, and the at least one insurance recommendation.

At least one of: a server, a processor, a microprocessor, a processing device may be configured to compensate for at least one of: a distorted image information, a missing image information, an obstructed image information. At least one of: a server, a processor, a microprocessor, a processing device may identify an image missing an information and instruct to at least one of: omit, process, reclassify the image. Identify an image with a distorted view of and instruct to at least one of: omit, process, reclassify the image. It should be understood, that a server, a processor, a processing device may also be configured to compensate for at least one of: a distorted image information, a missing image information, an obstructed image information on an image. Wherein an image includes a patient image.

Furthermore, a cluster analysis of the correlated patient information or a correlation dataset may be processed with a cluster dataset to produce additional information. Moreover, the correlated patient information or correlation dataset may be provided to an e-commerce company, an insurance company, a bioinformatics company, a business, a ML company, a cloud based company. The correlated patient information or correlation dataset may also be provided to a user or provider as a diagnostic aid.

The following will discuss the processing of at least one insurance data associated with at least one AI processed image. Processing at least one insurance data associated with at least one AI processed image with a non-described algorithm and/or a non-described data may disrupt the invention's ability to generate an insurance recommendation. The invention will be programed to omit non-described algorithms and/or non-described data associated with at least one insurance data associated with at least one AI processed image. Wherein at least one of: a server, a processor, a microprocessor, a processing device may replace at least one of: a non-described algorithm, a non-described data with at least of: a described algorithm, a described data and continue processing. At least one of: a described algorithm, a described data includes at least one of: the anatomy identification, the pathology identification. Wherein a described algorithm includes a described identification. Wherein the described identification includes at least one of: the anatomy identification, the pathology identification. Wherein a non-described identification may be replaced with a described identification. Wherein at least one of: a server, a processor, a microprocessor, a processing device may be programed to omit at least one of: a non-described algorithm, a non-described data associated with at least one insurance data associated with at least one AI processed image and replace with at least one of: a described algorithm, a described data and continue processing. A described data may include: at least one of: an American Dental Association (ADA) code, a date, an insurance claim identifier, an insurance claim number, an insurance claim, multiple or duplicate claims, a national provider identification number for a provider or an institution, a provider's license number, an age, a first name, a gender, a middle initial, a last name, a date of birth, a zip code, an address, a cell phone number, a land line number, a current medication, a previous medication, a social security number, a marital status, an insurance, an e-commerce consumer's insurance identification number, a change of insurance, a change of employment, a change of zip code, a change of the previous medication, a change of the marital status, a change of the gender, a first name, a middle name, a last name. A described algorithm may include at least one of: the anatomy identification, the pathology identification. Hence the processor is configured to omit processing a non-described data associated with the at least one insurance data associated with the at least one AI processed image. Wherein at least one of: a non-described algorithm, a non-described data may be replaced with at least one of: a described algorithm, a described data.

Processing at least one AI processed image associated with a non-described algorithm and/or a non-described data may disrupt the invention's ability to generate an insurance recommendation. Wherein at least one AI processed image may be a patient image. The invention will be programed to omit non-described algorithms and/or non-described data associated with at least one AI processed image. Wherein at least one of: a server, a processor, a microprocessor, a processing device may replace at least one of: a non-described algorithm, a non-described data with at least one of: a described algorithm, a described data and continue processing. A described algorithm includes at least one of: the anatomy identification, the pathology identification. Wherein a described algorithm may include a described identification. Wherein the described identification includes at least one of: the anatomy identification, the pathology identification. Wherein the processor is configured to omit processing the at least one AI processed image associated with a non-described identification. Wherein a non-described identification may be replaced with a described identification. Wherein at least one of: a server, a processor, a microprocessor, a processing device will be programed to omit at least one of: a non-described algorithm, a non-described data associated with at least one AI processed image and replace with a described algorithm and continue processing.

Further, processing at least one insurance data associated with a non-described algorithm and/or a non-described data may disrupt the invention's ability to generate an insurance recommendation. Wherein at least one of: a server, a processor, a microprocessor, a processing device will be programed to omit a non-described algorithm and/or a non-described data associated with at least one insurance data and replace with a described data and continue processing. The invention may continue to replace multiple non-described at least one insurance data and continue processing. At least one of: a server, a processor, a microprocessor, a processing device is configured to omit processing at least one insurance data with at least one of: a non-described algorithm, a non-described data. Wherein a non-described algorithm or a non-described data may include one or more of: a patient image set or image set with a label, object sub-types, confidence scores, a probability value of an image class, an image class vector, an image class space, a field of view label, a shallow hash neural network, a hash neural network, laboratory records of a patient, a laboratory test data, unified formats of lab test data, lab data off different formats, a computer code, a computer data. Wherein a lab is a laboratory. Wherein a label identifies a region of a particular anatomic structure. At least one of: a server, a processor, a microprocessor, a processing device may replace the omited processing with another data or algorithm and continue processing. At least one of: a server, a processor, a microprocessor, a processing device may identify a discrepancy is at least one of: a non-described algorithm, a non-described data. Further, at least one of: a server, a processor, a microprocessor, a processing device is configured to omit processing on an augmented reality display and replaced with a graphic user interphase (GUI) and/or user interphase (UI).

A described data may include: at least one of: an American Dental Association (ADA) code, a date, an insurance claim identifier, an insurance claim number, an insurance claim, multiple or duplicate claims, a national provider identification number for a provider or an institution, a provider's license number, an age, a first name, a gender, a middle initial, a last name, a date of birth, a zip code, an address, a cell phone number, a land line number, a current medication, a previous medication, a social security number, a marital status, an insurance, an e-commerce consumer's insurance identification number, a change of insurance, a change of employment, a change of zip code, a change of the previous medication, a change of the marital status, a change of the gender, a first name, a middle name, and a last name. Wherein the processor is configured to omit processing a non-described the at least one insurance data associated with the at least one AI processed image.

It is an object of the embodiments of the present invention to provide AI processing of Images to Generate an Insurance Recommendation.

It is an object of the embodiments of the present invention to determine, using to receive, using at least one processing device, at least one AI processed image associated with at least one insurance data, wherein the one AI processed image is based on a first AI model which includes at least one of: an anatomy identification, a pathology identification.

It is an object of the embodiments of the present invention to compare, using the at least one processing device, the at least one AI processed image associated with the at least one insurance data with a second AI model based on at least one of: the anatomy dataset, the pathology dataset, and the insurance dataset, wherein the second AI model may generate a weighted probability ratio of at least one of: an anatomy identification, a pathology identification found in the at least one AI processed image. Wherein the second AI model is further configured for determining at least one insurance recommendation based on the weighted probability ratio of at least one of: the anatomy identification, the pathology identification found in the at least one AI processed image associated with the at least one insurance data.

It is an object of the embodiments of the present invention to store, using a storage device at least one of: the at least one insurance data, the at least one AI processed image, the weighted probability ratio of at least one of: the anatomy identification, the pathology identification found in the at least one AI processed image, and the at least one insurance recommendation.

It is an object of the embodiments of the present invention to transmit, using the at least one processing device at least one of: the at least one insurance data, the at least one AI processed image, the weighted probability ratio of at least one of: the anatomy identification, the pathology identification found in the at least one AI processed image, and the at least one insurance recommendation.

These and other features, aspects and advantages of the present invention will become better understood with reference to the following drawings, descriptions and claims.

### BRIEF DESCRIPTION OF THE DRAWINGS

FIG. 1 shows a display diagram illustrating the mechanism that provides for the processing of at least one AI processed image associated with at least one insurance data for generating an insurance recommendation based on AI image processing.

FIG. 2 shows a display diagram illustrating the generation of an insurance recommendation may include at least one of: a medical insurance recommendation, a dental insurance recommendation.

FIG. 3 shows a display diagram which uses a processing device to verify compliance with a regulatory policy.

FIG. 4 shows a display diagram illustrating a mechanism that provides for the processing of at least one insurance data associated with at least one AI processed image for generating an insurance recommendation based on AI image processing and a user adjustable weighted probability ratio.

FIG. 5 is a block diagram of an example computing device, which may include at least one of: a server, a processor, a microprocessor, a processing device which may be used to provide at least one AI processed image associated with at least one insurance data to generate an insurance recommendation according to an embodiment of the invention.

### DESCRIPTION OF THE PREFERRED EMBODIMENTS

The preferred embodiments of the present invention will now be described with reference to the drawings. Examples of AI Processing of Images to Generating an Insurance Recommendation are discussed in this section. Identical elements in the various figures are identified with the same reference numerals.

Reference will now be made in detail to each embodiment of the present invention. Such embodiments are provided by way of explanation of the present invention, which are not intended to be limited thereto. In fact, those of ordinary skill in the art may appreciate upon reading the present specification and viewing the present drawings that various modifications and variations may be made thereto.

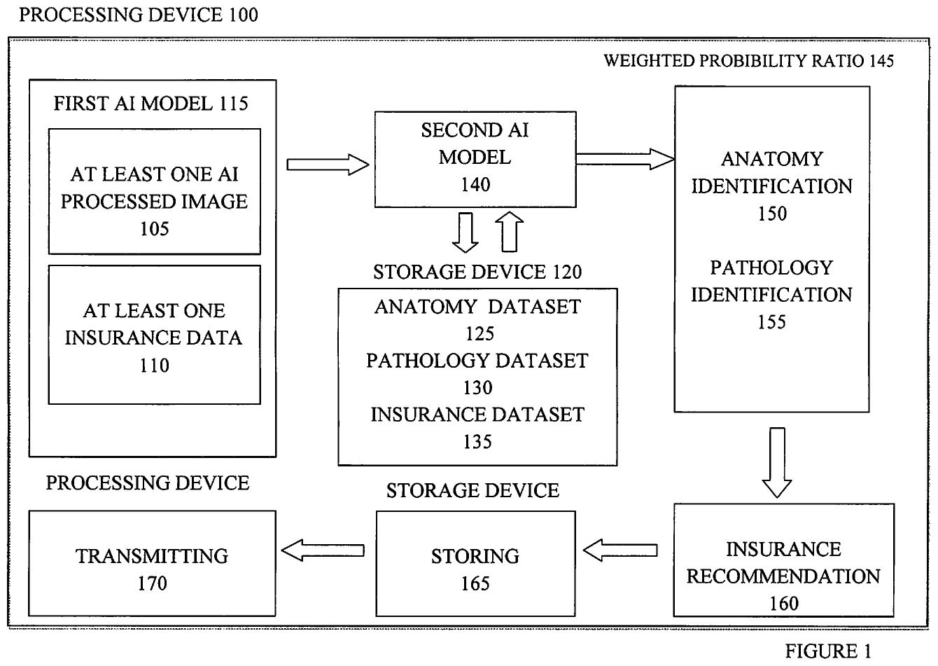

FIG. 1 shows a display diagram illustrating the mechanism that provides for the processing of AI images to generating an insurance recommendation. The diagram shows a method of generating an insurance recommendation based on artificial intelligence (AI) image processing, wherein the method comprising: receiving, using at least one processing device (100), at least one AI processed image (105) associated with at least one insurance data (110). Wherein the at least one AI processed image may be obtained from an image capture device. Wherein a processing device may include one or more of: a server, a processor, a microprocessor, a processing device. Wherein the one AI processed image is based on a first AI model which includes at least one of: an anatomy identification, a pathology identification. Retrieving, using a storage device (120), at least one of: an anatomy dataset (125), a pathology dataset (130), and an insurance dataset (135). Comparing, using the at least one processing device, the at least one AI processed image associated with the at least one insurance data with a second AI model (140) based on at least one of: the anatomy dataset (125), the pathology dataset (130), and the insurance dataset (135). Wherein the second AI model may generate a weighted probability ratio (145) of at least one of: an anatomy identification (150), a pathology identification (155) found in the at least one AI processed image. Wherein the second AI model is further configured for determining at least one insurance recommendation (160) based on the weighted probability ratio of at least one of: the anatomy identification, the pathology identification found in the at least one AI processed image associated with the at least one insurance data. Storing (165), using the storage device at least one of: the at least one insurance data, the at least one AI processed image, the weighted probability ratio of at least one of: the anatomy identification, the pathology identification found in the at least one AI processed image, and the at least one insurance recommendation. Transmitting (170), using the at least one processing device at least one of: the at least one insurance data, the at least one AI processed image, the weighted probability ratio of at least one of: the anatomy identification, the pathology identification found in the at least one AI processed image, and the at least one insurance recommendation.

The method in FIG. 1, wherein the at least one processor device may be configured to only process a described identification associated with the at least one AI processed image. Wherein the described identification includes at least one of: the anatomy identification, the pathology identification. Wherein the processor is configured to omit processing the at least one AI processed image associated with a non-described identification. Wherein a non-described identification may be replaced with a described identification.

The method in FIG. 1 may be further configured, wherein the at least one processor device may be configured to only process a described the at least one insurance data. Wherein the described the at least one insurance data may include at least one of: an American Dental Association (ADA) code, a date, an insurance claim identifier, an insurance claim number, an insurance claim, multiple or duplicate claims, a national provider identification number for a provider or an institution, a provider's license number, an age, a first name, a gender, a middle initial, a last name, a date of birth, a zip code, an address, a cell phone number, a land line number, a current medication, a previous medication, a social security number, a marital status, an insurance, an e-commerce consumer's insurance identification number, a change of insurance, a change of employment, a change of zip code, a change of the previous medication, a change of the marital status, a change of the gender, a first name, a middle name, and a last name. Wherein the processor is configured to omit processing a non-described the at least one insurance data associated with the at least one AI processed image. Wherein a non-described at least one insurance data may be replaced with the described the at least one insurance data.

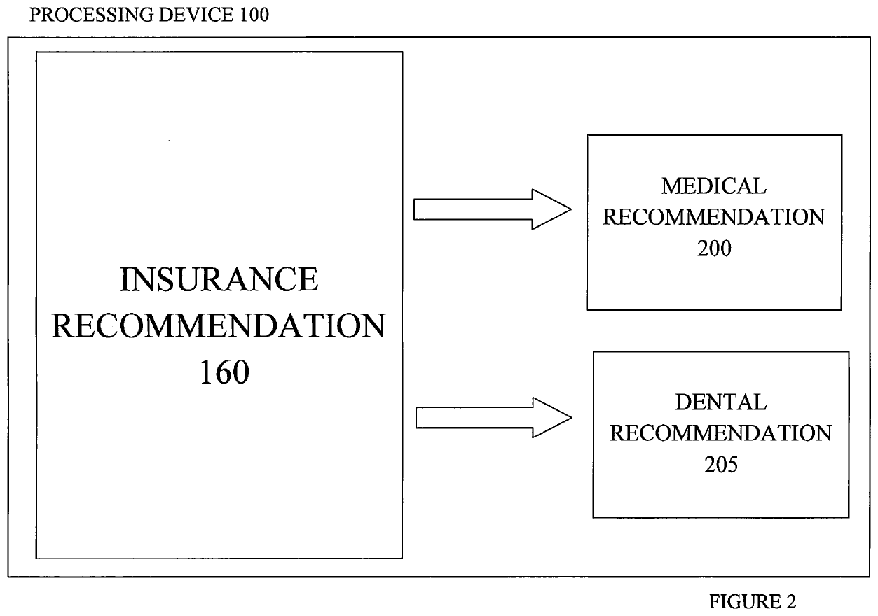

The method of FIG. 1 may also be further configured, wherein the at least one processor may format the at least one AI processed image based on a transaction processed by a user. Merge the at least one AI processed image with a transaction processed by a user into a correlation dataset. Identify and correct a discrepancy between the at least one AI processed image with a transaction processed by a user and provide to the correlation dataset. Comparing, using the processing device, the at least one insurance data and the correlation dataset with a second AI model based on at least one of: the anatomy dataset, the pathology dataset, and the insurance dataset. Wherein the second AI model may generate a weighted probability ratio of at least one of: the anatomy identification, the pathology identification found in the at least one AI processed image. FIG. 2 demonstrates wherein the generating of an insurance recommendation (160) may include at least one of: a medical insurance recommendation (200), a dental insurance recommendation (205).

Another aspect of the method of FIG. 1, wherein a weighted probability ratio may include at least one of: a weighted likelihood score, a probabilistic weighting factor, a weighted likelihood measure, a probability weighting coefficient, a weighted probability assessment, a weighted likelihood index, a probabilistic weighting ratio, a weighted likelihood quotient, a weighted likelihood ratio, a probabilistic weighting index, a weighted likelihood coefficient, a probability weighting score, a weighted probability factor, a probabilistic weighting measure, a weighted likelihood proportion, a probability weighting quotient, a weighted probability index, and an adjustable weighted probability ratio. Further, the method of FIG. 1, wherein the weighted probability ratio threshold may use an adjustable weighted probability ratio to determine and identified at least one of: the anatomy identification, the pathology identification found in the at least one AI processed image. Further the method of claim 1, wherein the at least one processing device may process an instruction in any order.

Another embodiment of the AI Processing of Images to Generate an Insurance Recommendation will now be discussed. A method of generating an insurance recommendation based on artificial intelligence (AI) image processing, wherein the method comprising: receive, using a processing device, at least one insurance data associated with at least one AI processed image. Wherein the processing device may execute an instruction in any order. Wherein the one AI processed image is based on a first AI model which includes at least one of: an anatomy identification, a pathology identification. Retrieve, using a storage device, at least one of: an anatomy dataset, a pathology dataset, an insurance dataset. Compare, using the processing device, the at least one insurance data and the at least one AI processed image with a second AI model based on at least one of: the anatomy dataset, the pathology dataset, and the insurance dataset. Wherein the second AI model may generate a weighted probability ratio of at least one of: the anatomy identification, the pathology identification found in the at least one AI processed image. Using the processing device, the second AI model is configured for determining the at least one insurance recommendation based on the weighted probability ratio of at least one of: an anatomy identification, a pathology identification found in the at least one AI processed image and associate with the at least one insurance data. Store, using the storage device at least one of: the at least one insurance data, the at least one AI processed image, the weighted probability ratio of at least one of: the anatomy identification, the pathology identification found in the at least one AI processed image, and the at least one insurance recommendation. And transmit, using the processing device at least one of: the at least one insurance data, the at least one AI processed image, the weighted probability ratio of at least one of: the anatomy identification, the pathology identification found in the at least one AI processed image, and the at least one insurance recommendation.

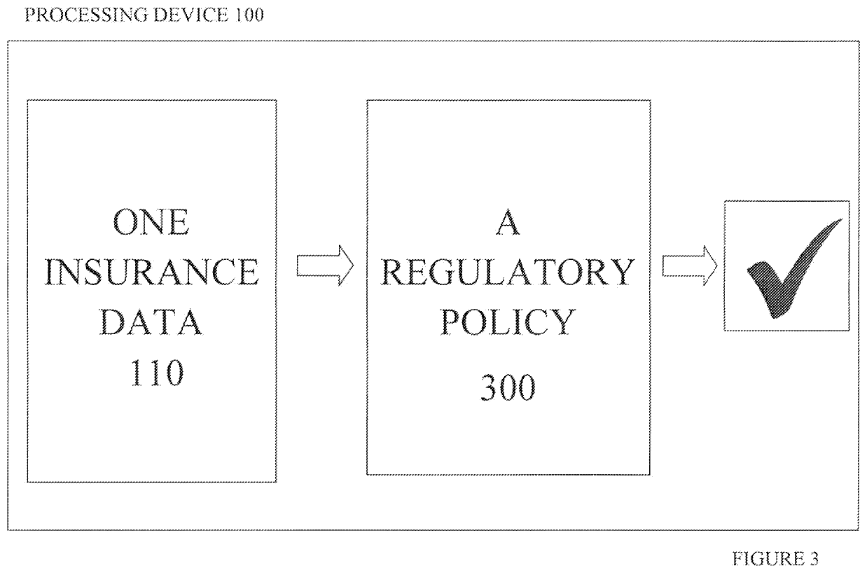

In FIG. 3, the method is further configured to use a processing device (100) to verify compliance with a regulatory policy (300). Wherein the at least one insurance data (110) may be verified compliant with a regulatory policy (300). Another aspect on the invention is that one insurance data may be obtained from multiple sources. Wherein the at least one insurance data may be obtained from one or more of: a user, a provider, an e-commerce organization, a machine learning entity, a cloud based storage, an algorithm, a bioinformatics dataset, a business, and an insurance dataset.

The invention is further configured to calculate a weighted probability ratio. Wherein the processing device may calculate the weighted probability ratio using an adjustable weighted average. Another aspect of the invention is the ability for the processing device to apply image preprocessing techniques to the at least one AI processed image before processing with a second AI model. The applied image preprocessing techniques may be adjusted by a user or be automated by either a ML algorithm or an AL algorithm. Further, the method is configured to compensate for a distorted or missing information. Wherein the processing device is configured to compensate for at least one of: a distorted information, a missing image, an obstructed information on the at least one AI processed image. Wherein the one AI processed image may be preprocessed using a combination of machine learning anatomy identification and pathology identification algorithms.

The invention may use the processing device to identify at least one of: the weighted probability ratio of at least one of: the anatomy identification, the pathology identification found in the at least one AI processed image, and the at least one insurance recommendation using a combination of ML anatomy and pathology algorithms. Further, the second AI model weighted probability ratio may be processed with a multiple AI model to determine at least one insurance recommendation. Wherein the multiple AI model may be based on the weighted probability ratio of at least one of: the anatomy identification, the pathology identification. Another important aspect of the invention is the ability for a user or an AI algorithm may adjust the weighted probability ratio. Wherein the processing device may calculate the weighted probability ratio using a user adjustable weighted probability ratio. Wherein the processing device may calculate the weighted probability ratio using an AI algorithm adjustable weighted probability ratio. Another aspect of the invention comprises a user interface (UI) and/or a graphic used interphase (GUI) for displaying at least one of: the at least one insurance data, the at least one AI processed image, the weighted probability ratio of at least one of: an anatomy identification, a pathology identification found in the at least one AI processed image, the at least one insurance recommendation. Wherein a UI or a GUI may be configured with at least one of: a server, a processor, microprocessor, a processing device.

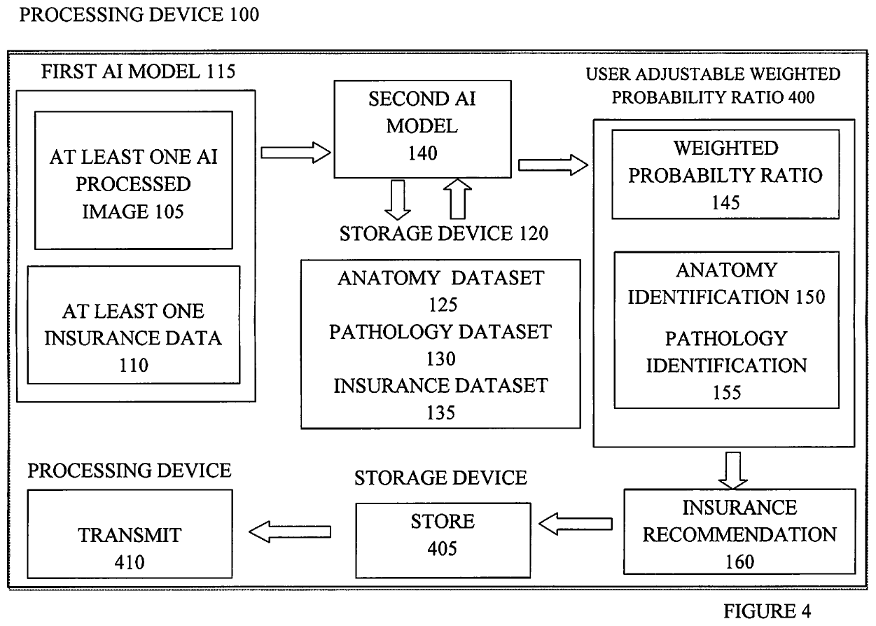

FIG. 4 shows yet another embodiment of the invention. AI Processing of Images to Generate an Insurance Recommendation will now be discussed. A system of generating an insurance recommendation based on artificial intelligence (AI) image processing is as follows. Wherein the system comprising: receive, using at least one processing device (100), at least one insurance data associated (110) with at least one AI processed image (105). Wherein the at least one insurance data (110) may be a patient data. Wherein the at least one AI processed image (105) may be a patient image. Wherein the at least one AI processed image (105) is based on a first AI model which includes at least one of: an anatomy identification, a pathology identification. Retrieve, using a storage device (120), at least one of: an anatomy dataset (125), a pathology dataset (130), an insurance dataset (135). Compare, using the at least one processing device, the at least one insurance data (110) and the at least one AI processed image (105) with a second AI model based on at least one of: an anatomy dataset (125), a pathology dataset (130), and an insurance dataset (135). Generate using the second AI model a weighted probability ratio (145), with a user adjustable weighted probability ratio (400) to produce at least one of: a weighted probability ratio of an anatomy identification (150), a weighted probability ratio of a pathology identification (155) found in the at least one AI processed image (105). Generate using the second AI model at least one insurance recommendation (160) based on the weighted probability ratio (145) of at least one of: a weighted probability ratio of an anatomy identification, a weighted probability ratio of a pathology identification found in the at least one insurance data (110) associated with the at least one AI processed image (105). Store, using the storage device (405) at least one of: the at least one insurance data, the at least one AI processed image, the weighted probability ratio of at least one of: the weighted probability ratio of an anatomy identification, the weighted probability ratio of a pathology identification found in the at least one AI processed image, and the at least one insurance recommendation. Transmit (410), using the at least one processing device at least one of: the at least one insurance data, the at least one AI processed image, the weighted probability ratio of at least one of: the weighted probability ratio of an anatomy identification, the weighted probability ratio of a pathology identification found in the at least one AI processed image, and the at least one insurance recommendation. The invention is further configured: wherein the user adjustable weighted probability ratio may be configured to identify a discrepancy in the at least one AI processed image.

The system or method of may be configured to use multiple measuring tool. Wherein the weighted probability ratio may use an adjustable weighted probability ratio threshold to determine and identified at least one of: the anatomy identification, the pathology identification found in the at least one AI processed image. Further, the weighted probability ratio may include at least one of: a weighted likelihood score, a probabilistic weighting factor, a weighted likelihood measure, a probability weighting coefficient, a weighted probability assessment, a weighted likelihood index, a probabilistic weighting ratio, a weighted likelihood quotient, a weighted likelihood ratio, a probabilistic weighting index, a weighted likelihood coefficient, a probability weighting score, a weighted probability factor, a probabilistic weighting measure, a weighted likelihood proportion, a probability weighting quotient, a weighted probability index, and an adjustable weighted probability ratio.

Another aspect field of the invention relates to a system or method of using Artificial Intelligence (AI) processing of images to generate an insurance recommendation. The invention is configured to receive a preprocessed AI image associated with an insurance data and then compare and process the preprocessed AI image with a separate second or multiple AI algorithm. The invention is further configured to use a user adjustable probability ratio to identify at least one of: an anatomy identification, a pathology identification. The second or multiple processing of the preprocessed image may occur at an offsite location, such an insurance platform or the cloud. The second or multiple AI algorithm may generate an insurance recommendation based on the preprocessed AI image. The system or method comprising: receive, using a processing device, at least one insurance data associated with at least one preprocessed AI processed image. Wherein the processing device may execute an instruction in any order. Wherein the one preprocessed AI processed image is based on a first AI model which includes at least one of: an anatomy identification, a pathology identification. Retrieve, using a storage device, at least one of: an anatomy dataset, a pathology dataset, an insurance dataset. Compare, using the processing device, the at least one insurance data and the at least one preprocessed AI processed image with a second AI model based on at least one of: the anatomy dataset, the pathology dataset, and the insurance dataset. Wherein the second AI model may generate a weighted probability ratio of at least one of: the anatomy identification, the pathology identification found in the at least one AI processed image. Using the processing device, the second AI model is configured for determining the at least one insurance recommendation based on the weighted probability ratio of at least one of: an anatomy identification, a pathology identification found in the at least one preprocessed AI processed image and associate with the at least one insurance data. The system or method may be further configured to use a multiple AI model to continue processing the second AI model generation of a weighted probably ration of at least one of: the anatomy identification, the pathology identification to determine at least one insurance recommendation.

Store, using the storage device at least one of: the at least one insurance data, the at least one preprocessed AI processed image, the weighted probability ratio of at least one of: the anatomy identification, the pathology identification found in the at least one AI processed image, and the at least one insurance recommendation. And transmit, using the processing device at least one of: the at least one insurance data, the at least one preprocessed AI processed image, the weighted probability ratio of at least one of: the anatomy identification, the pathology identification found in the at least one AI processed image, and the at least one insurance recommendation.

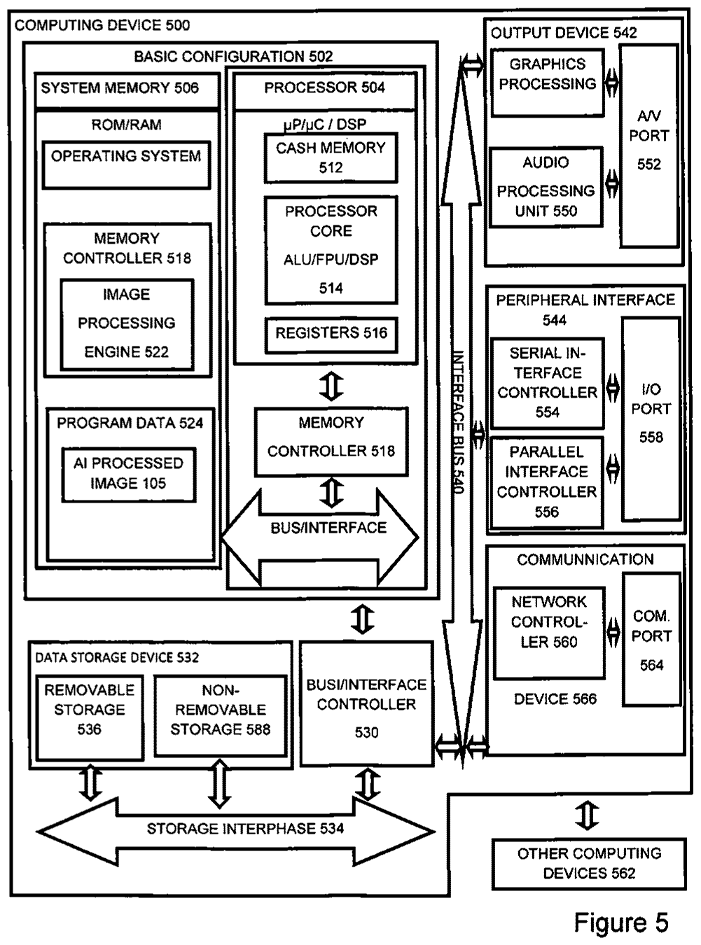

FIG. 5 is an example of a computing device 500. Wherein a computing device may include at least one of: a server, a processor, a microprocessor, a processing device. Depending on the desired configuration, the processor 504 may be of any type, including but not limited to a server, a processor, a microprocessor (uP), a processing device, a microcontroller (uC), a digital signal processor (DSP), or any combination thereof. The processor 504 may include one or more levels of caching, such as a level cache memory 512, one or more processor cores 514, and registers 516. The example processor cores 514 may (each) include an arithmetic logic unit (ALU), a floating-point unit (FPU), a digital signal processing core (DSP Core), a graphics processing unit (GPU), or any combination thereof. An example memory controller 518 may also be used with the processor 504, or in some implementations, the memory controller 518 may be an internal part of the processor 504.

Depending on the desired configuration, the system memory 506 may be of any type including but not limited to volatile memory (such as RAM), non-volatile memory (such as ROM, flash memory, etc.), or any combination thereof. The system memory 506 may store and provide an operating system 520, with at least one of: a server, a processor, a microprocessor, a processing device and a program data 524. At least one of: a server, a processor, a microprocessor, a processing device may include components such as an image processing engine 522. The image processing engine 522 may execute the instructions and processes associated with at least one of: a processor, a microprocessor, a processing device, a server. In an example scenario, the image processing engine 522 may receive at least one AI processed image associated with at least one insurance data from a user or provider.

Input to and output out of at least one of: a server, a processor, a microprocessor, a processing device may be transmitted through a communication device 566 that may be communicatively coupled to the computing device 500. A computing device 500 may include at least one of: a server, a processor, a microprocessor, a processing device. The communication device 566 may provide wired and/or wireless communication. The program data 524 may also include, among other data, at least one AI processed image associated with at least one insurance data for a user or provider, or the like, as described herein. The at least one AI processed image may include at least one of: an anatomy identification, a pathology identification.

The computing device 500 may have additional features or functionality, and additional interfaces to facilitate communications between the basic configuration 502 and any desired devices and interfaces. For example, a bus/interface controller 530 may be used to facilitate communications between the basic configuration 502 and one or more data storage devices 532 via a storage interface bus 534. The data storage devices 532 may be one or more removable storage devices 536, one or more non-removable storage devices 538, a cloud storage device, or a combination thereof. Examples of the removable storage and the non-removable storage devices may include magnetic disk devices, such as flexible disk drives and hard-disk drives (HDDs), optical disk drives such as compact disk (CD) drives or digital versatile disk (DVD) drives, solid state drives (SSDs), tape drives, flash memory, cloud based storage, a cloud computing platform providing a storage service, an open or a closed source platform providing a storage service, a virtual private network (VPN) providing a storage service, an ISO image disk, a cloud based storage service, a redundant array of independent disks (RAID), a USB based disk drive, a USB flash drive, a storage virtualization based storage service, a digital video service, a virtualized server providing a storage service, a super computer providing a storage service, a super computer parallel array providing a storage service, a medical practice management software providing a storage service, a medical digital image software providing a storage service, a dental practice management software providing a storage service, a dental digital image software providing a storage service, and/or all future embodiments. Example computer storage media may include volatile and nonvolatile, removable, and non-removable media implemented, cloud based storage in any method or technology for storage of information, such as computer-readable instructions, data structures, program modules, all future embodiments, or other data.

The system memory 506, the removable storage devices 536 and the non-removable storage devices 538 are examples of computer storage media. Computer storage media includes, but is not limited to, RAM, ROM, EEPROM, flash memory or other memory technology, cloud based memory, cloud based storage, CD-ROM, digital versatile disks (DVDs), solid state drives, or other optical storage, quantum memory, magnetic cassettes, magnetic tape, magnetic disk storage or other magnetic storage devices, or any other medium which may be used to store the desired information and which may be accessed by the computing device 500. Any such computer storage media may be part of the computing device 500.

The computing device 500 may also include an interface bus 540 for facilitating communication from various interface devices (for example, one or more output devices 542, one or more peripheral interfaces 544, and one or more communication devices 566) to the basic configuration 502 via the bus/interface controller 530. Some of the example output devices 542 include a graphics processing unit 548, GUI, UI, and an audio processing unit 550, which may be configured to communicate to various external devices such as a display, GUI, or speakers via one or more A/V ports 552. One or more example peripheral interfaces 544 may include a serial interface controller 554 or a parallel interface controller 556, which may be configured to communicate with external devices such as input devices (for example, keyboard, mouse, pen, voice input device, touch input device, etc.) or other peripheral devices (for example, printer, scanner, etc.) via one or more I/O ports 558. An example of the communication device(s) 566 includes a network controller 560, which may be arranged to facilitate communications with one or more other computing devices 562 over a network communication link via one or more communication ports 564. The one or more other computing devices 562 may include at least one of: a server, a processor, a microprocessor, a processing device, a computing device, and comparable devices.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) WiFi, Bluetooth, short range wireless interconnection, long range wireless interconnection, wireless networking technology, radio waves, light waves, any electromagnetic wave, and other wireless media. The term computer readable media as used herein may include both storage media and communication media.

The computing device 500 may be implemented as a part of a specialized server, mainframe, or similar computer, which includes any of the above functions. The computing device 500 may also be implemented on personal computer device(s) such as a laptop computer, a cell phone, and non-laptop computer configurations. Additionally, the computing device 500 may include specialized hardware such as an application-specific integrated circuit (ASIC), a field programmable gate array (FPGA), a programmable logic device (PLD), and/or a free form logic on an integrated circuit (IC), among others.

Additional example embodiments for a system or method to provide AI Processing of Images to Generate an Insurance Recommendation are as follows. The system or method can be implemented in any number of ways, including or having structures described herein. One such way may be by machine operations, of devices of the type described in the present disclosure. Wherein one or more of the individual operations of the system or the method may be performed in conjunction with one or more human operators. Hence performing some of the operations by individuals while other operations may be performed by algorithms. These human operators need not be collocated with each other, but each can be only with a machine that performs a portion of the program. In other embodiments, the human interaction can be automated such as by pre-selected criteria that may be machine automated.

The embodiments of the invention, example scenarios and schemas in FIGS. 1 through 5 are shown with specific components, data types, and configurations. Embodiments are not limited to systems according to these example configurations. The invention, AI Processing of Images to Generate an Insurance Recommendation may be implemented in configurations employing fewer or additional components in applications and user interfaces. Furthermore, the example schema and components shown in FIGS. 1 through 5 and their subcomponents may be implemented in a similar manner with other values using the principles described herein.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

1. A method of generating an insurance recommendation based on artificial intelligence (AI) image processing, wherein the method comprising:
receiving, using at least one processing device, at least one AI processed image associated with at least one insurance data;
wherein the one AI processed image is based on a first AI model which includes at least one of:
an anatomy identification, a pathology identification;
retrieving, using a storage device, at least one of: an anatomy dataset, a pathology dataset, and an insurance dataset;
comparing, using the at least one processing device, the at least one AI processed image associated with the at least one insurance data with a second AI model based on at least one of:
the anatomy dataset, the pathology dataset, and the insurance dataset;
wherein the second AI model may generate a weighted probability ratio of at least one of: an anatomy identification, a pathology identification found in the at least one AI processed image;
wherein the second AI model is further configured for determining at least one insurance recommendation based on the weighted probability ratio of at least one of: the anatomy identification, the pathology identification found in the at least one AI processed image associated with the at least one insurance data;
storing, using the storage device at least one of: the at least one insurance data, the at least one AI processed image, the weighted probability ratio of at least one of: the anatomy identification, the pathology identification found in the at least one AI processed image, and the at least one insurance recommendation; and
transmitting, using the at least one processing device at least one of: the at least one insurance data, the at least one AI processed image, the weighted probability ratio of at least one of: the anatomy identification, the pathology identification found in the at least one AI processed image, and the at least one insurance recommendation.

2. The method of claim 1, wherein the at least one processor device may be configured to only process a described identification associated with the at least one AI processed image;
wherein the described identification includes at least one of: the anatomy identification, the pathology identification;
wherein the processor is configured to omit processing the at least one AI processed image associated with a non-described identification;
wherein a non-described identification may be replaced with a described identification.

3. The method of claim 1, wherein the at least one processor device may be configured to only process a described the at least one insurance data;
wherein the described the at least one insurance data may include at least one of: an American Dental Association (ADA) code, a date, an insurance claim identifier, an insurance claim number, an insurance claim, multiple or duplicate claims, a national provider identification number for a provider or an institution, a provider's state license number, an age, a first name, a gender, a middle initial, a last name, a date of birth, a zip code, an address, a cell phone number, a land line number, a current medication, a previous medication, a social security number, a marital status, an insurance, an e-commerce consumer's insurance identification number, a change of insurance, a change of employment, a change of zip code, a change of the previous medication, a change of the marital status, a change of the gender, a first name, a middle name, and a last name;
wherein the processor is configured to omit processing a non-described the at least one insurance data associated with the at least one AI processed image;
wherein a non-described at least one insurance data may be replaced with the described the at least one insurance data.

4. The method of claim 1, wherein the at least one processor may format the at least one AI processed image based on a transaction processed by a user;
merge the at least one AI processed image with a transaction processed by the user into a correlation dataset;
identify and correct a discrepancy between the at least one AI processed image with a transaction processed by the user and provide to the correlation dataset;
comparing, using the processing device, the at least one insurance data and the correlation dataset with a second AI model based on at least one of: the anatomy dataset, the pathology dataset, and the insurance dataset;
wherein the second AI model may generate the weighted probability ratio of at least one of: the anatomy identification, the pathology identification found in the at least one AI processed image.

5. The method of claim 1, wherein the weighted probability ratio may include at least one of: a weighted likelihood score, a probabilistic weighting factor, a weighted likelihood measure, a probability weighting coefficient, a weighted probability assessment, a weighted likelihood index, a probabilistic weighting ratio, a weighted likelihood quotient, a weighted likelihood ratio, a probabilistic weighting index, a weighted likelihood coefficient, a probability weighting score, a weighted probability factor, a probabilistic weighting measure, a weighted likelihood proportion, a probability weighting quotient, a weighted probability index, and an adjustable weighted probability ratio.

6. The method of claim 1, wherein the at least one processing device may process an instruction in any order;
wherein the generating of an insurance recommendation may include at least one of: a medical insurance recommendation, a dental insurance recommendation;
wherein the at least one AI processed image associated with at least one insurance data may be a preprocessed at least one AI image associated with at least one insurance data.

7. The method of claim 1, wherein the weighted probability ratio may use an adjustable weighted probability ratio threshold to determine and identified at least one of: the anatomy identification, the pathology identification found in the at least one AI processed image.

8. A method of generating an insurance recommendation based on artificial intelligence (AI) image processing, wherein the method comprising:
receive, using a processing device, at least one insurance data associated with at least one AI processed image;
wherein the processing device may execute an instruction in any order;
wherein the one AI processed image is based on a first AI model which includes at least one of:
an anatomy identification, a pathology identification;
retrieve, using a storage device, at least one of: an anatomy dataset a pathology, dataset, an insurance dataset;
compare, using the processing device, the at least one insurance data and the at least one AI processed image with a second AI model based on at least one of: the anatomy dataset, the pathology dataset, and the insurance dataset;
wherein the second AI model may generate a weighted probability ratio of at least one of: the anatomy identification, the pathology identification found in the at least one AI processed image; using the processing device, the second AI model is configured for determining the at least one insurance recommendation based on the weighted probability ratio of at least one of: an anatomy identification, a pathology identification found in the at least one insurance data associated with the at least one AI processed image;
store, using the storage device at least one of: the at least one insurance data, the at least one AI processed image, the weighted probability ratio of at least one of: the anatomy identification, the pathology identification found in the at least one AI processed image, and the at least one insurance recommendation; and
transmit, using the processing device at least one of: the at least one insurance data, the at least one AI processed image, the weighted probability ratio of at least one of: the anatomy identification, the pathology identification found in the at least one AI processed image, and the at least one insurance recommendation.

9. The method of claim 8, wherein the at least one insurance data may be verified compliant with a regulatory policy.

10. The method of claim 8, wherein the at least one insurance data may be obtained from one or more of: a user, a provider, an e-commerce organization, a machine learning entity, a cloud based storage, an algorithm, a bioinformatics dataset, a business, and an insurance dataset.

11. The method of claim 8, wherein the processing device may calculate the weighted probability ratio using an adjustable weighted average.

12. The method of claim 8, wherein the processing device may apply image preprocessing techniques to the at least one AI processed image before processing with a second AI model.

13. The method of claim 8, wherein the processing device is configured to compensate for at least one of: a distorted information, a missing image, and an obstructed information on the at least one AI processed image.

14. The method of claim 8, wherein the processing device identifies at least one of: the weighted probability ratio of at least one of: the anatomy identification, the pathology identification found in the at least one AI processed image, and the at least one insurance recommendation using a combination of ML anatomy and pathology algorithms.

15. The method of claim 8, further comprising a user interface for displaying at least one of:
the at least one insurance data, the at least one AI processed image, the weighted probability ratio of at least one of: an anatomy identification, a pathology identification found in the at least one AI processed image, and the at least one insurance recommendation.

16. The method of claim 8, wherein the one AI processed image may be preprocessed using a combination of machine learning anatomy identification and pathology identification algorithms;
wherein the at least one insurance data associated with at least one AI processed image may be an at least one insurance data associated with at least one preprocessed AI image.

17. The method of claim 8, wherein the second AI model weighted probability ratio may be processed with a multiple AI model to determine at least one insurance recommendation;
wherein the multiple AI model may be based on the weighted probability ratio of at least one of:
the anatomy identification, the pathology identification;
wherein the second AI model may be a machine learning algorithm;
wherein the multiple AI model may be a machine learning algorithm.

18. The method of claim 8, wherein the processing device may calculate the weighted probability ratio using a user adjustable weighted probability ratio.

19. A system of generating an insurance recommendation based on artificial intelligence (AI) image processing, wherein the system comprising:
receive, using at least one processing device, at least one insurance data associated with at least one AI processed image;
wherein the at least one AI processed image is based on a first AI model which includes at least one of: an anatomy identification, a pathology identification;
retrieve, using a storage device, at least one of: an anatomy dataset, a pathology dataset, an insurance dataset;
compare, using the at least one processing device, the at least one insurance data and the at least one AI processed image with a second AI model based on at least one of: an anatomy dataset, a pathology dataset, and an insurance dataset;
generate using the second AI model a weighted probability ratio, with a user adjustable weighted probability ratio to produce of at least one of: a weighted probability ratio of an anatomy identification, a weighted probability ratio of a pathology identification found in the at least one AI processed image;
generate using the second AI model at least one insurance recommendation based on the user adjustable weighted probability ratio of at least one of: a weighted probability ratio of an anatomy identification, a weighted probability ratio of a pathology identification found in the at least one insurance data associated with the at least one AI processed image;
store, using the storage device at least one of: the at least one insurance data, the at least one AI processed image, the weighted probability ratio of at least one of: the weighted probability ratio of an anatomy identification, the weighted probability ratio of a pathology identification found in the at least one AI processed image, and the at least one insurance recommendation; and
transmit, using the at least one processing device at least one of: the at least one insurance data, the at least one AI processed image, the weighted probability ratio of at least one of: the weighted probability ratio of an anatomy identification, the weighted probability ratio of a pathology identification found in the at least one AI processed image, and the at least one insurance recommendation.

20. The system of claim 19, wherein the user adjustable weighted probability ratio may be configured to identified a discrepancy in the at least one AI processed image.
